(19) United States
(12) Patent Application Publication
(10) Pub. No.: US 2009/0082596 A1
(43) Pub. Date: Mar. 26, 2009

(54) Process for the Preparation of Adamantanamines

(76) Inventor: Christian Pahick Schickaneder, Dresden (DE)

Correspondence Address: SANDOZ INC, 506 CARNEFIE CENTER, PRINCETON, NJ, 08540, US

(21) Appl. No.: 12/224,147

(22) PCT Filed: Feb. 20, 2007
(86) PCT No.: PCT/EP2007/001440

§ 371 Date: Aug. 19, 2008

(30) Foreign Application Priority Data
Feb. 21, 2006 (EP) 06003477

(51) Int. Cl. C07C 233/08; C07C 209/78; C07C 257/10

(52) U.S. Cl. 564/210; 564/460; 564/225

(57) ABSTRACT

The invention relates to a process for preparing certain adamantanamines, of formula

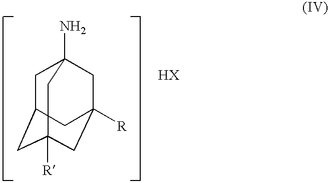

wherein R, R′ are each methyl and X is halogen, to intermediates used in the process, and to processes for preparing such intermediates.

The invention relates to a process for preparing certain adamantanamines, to intermediates used in the process, and to processes for preparing such intermediates.

Adamantanamines are known for long as a class of valuable pharmaceuticals. For example, Memantine hydrochloride (1-amino-3,5-dimethyladamantane hydrochloride, formula IVa)

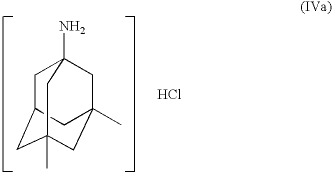

is an uncompetitive NMDA(N-methyl-D-aspartate) receptor antagonist, which is used as a new treatment for Alzheimer's disease. Memantine hydrochloride launched in the market as Axura® is the first AD therapeutic compound which acts via this mechanism of action. Memantine has been marketed for more than 20 years in Germany for the treatment of spasticity and dementia syndrome.

Amantadine hydrochloride (1-aminoadamantane, formula IVb)

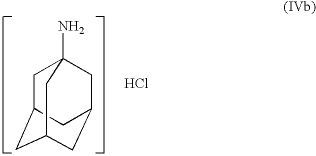

is an antiviral used to treat certain influenza infections (type A). The compound is also an antidyskinetic used to treat Parkinson's disease.

U.S. Pat. No. 3,391,142 describes a process for preparing Memantine hydrochloride starting from 1-bromo-3,5-dimethyladamatane, which is reacted with acetonitrile in concentrated sulphuric acid to yield the acetamido derivative, which is then cleaved under basic hydrolysis conditions.

According to WO 01/053234 adamantane is converted into N-(1-adamantyl)acetamide in a reaction medium containing dry acetonitrile, fluorine, and a lewis acid. This compound can be cleaved under acidic or basic conditions yielding amino adamantane.

To avoid a reaction with highly toxic HCN, several processes describe the reaction of dimethyladamantanes with urea or formamide (WO 05/023753, RU 2246482, DE 23184461, EP 0392059, CZ 288445).

There are a number of problems encountered with the conventional processes for preparing adamantanamines. The yields of at least some of the steps are low, the reaction time is relatively high, hazardous reaction conditions and/or solvents are required and/or the use of some solvents causes difficulties in purification of the final product.

Therefore there is a need for an improved process for preparing highly pure adamantanamines, in particular Memantine hydrochloride or Amantadine hydrochloride, which overcomes the above-mentioned problems and thereby provides a process which is economic and viable on a commercial scale.

Thus, the present invention in is one aspect directed to a process for the manufacture of an adamantanamine of formula

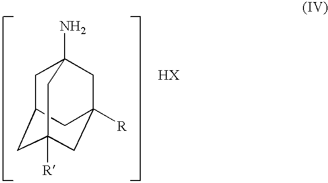

wherein R and R′ are each methyl and X is halogen, which comprises
(i) reacting a compound of formula

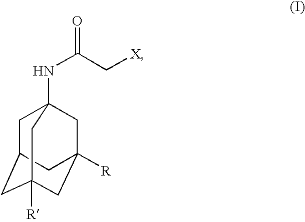

wherein R, R′ and X are each as defined above, with thiourea;
(ii) subjecting the resulting compound of formula

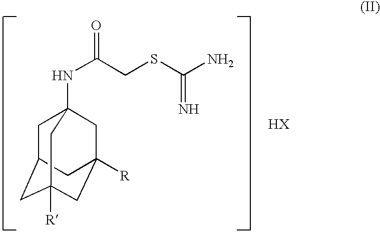

to an acid treatment and
(iii) isolating the resulting adamantanamine or a hydrohalogenide thereof.

In general, X can be any halogen, like chlorine, bromine, iodine. In a special embodiment of the present invention, X is chlorine or bromine.

The process of the invention is especially suited for the manufacture of Memantine hydrohalogenides, in particular Memantine hydrochloride, by using a compound of the formula (I), wherein R and R′ are each methyl and X is halogen, in particular chlorine or bromine, as the starting material.

Another aspect of the present invention is a compound of formula (I),

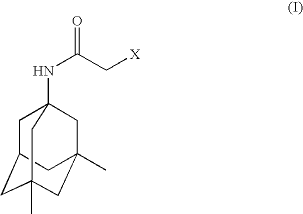

wherein R and R′ are each methyl and X is chlorine or bromine.

Compounds of formula (I) can be obtained by reacting a compound of formula

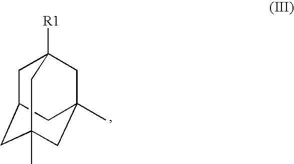

wherein R1 is hydroxy or halogen, with a haloacetonitrile X—CH₂—CN, wherein X is halogen, in particular chlorine or bromine, in an acidic medium.

R1 is, for example hydroxy, bromine or chlorine, in particular hydroxyl or bromine. Throughout this application X is, for example, bromine or chlorine and in particular chlorine. The compounds of formula (III) are known or may be obtained according to methods known per se.

The acidic medium used for the reaction of the compound of formula (III) with the haloacetonitrile, in particular chloroacetonitrile, suitably comprises a strong mineral acid, in particular sulphuric acid. Further optional components of the acidic medium comprise one or more solvents, for example an organic acid, for example propionic acid or in particular acetic acid; and/or a polar aprotic solvent, for example N,N-dimethyl formamide (DMF); and a metal salt catalyst, for example iron(III) sulfate.

One embodiments of the present invention comprise the reaction of 1-hydroxy-3,5-dimethyl adamantane (compound of formula (III), wherein R is hydroxyl) with chloroacetonitrile in a solution comprising sulphuric acid; an organic acid, in particular acetic acid; and optionally DMF.

Another embodiment comprises the reaction of 1-bromo-3,5-dimethyl adamantane (compound of formula (III), wherein R is bromo) with chloroacetonitrile in a solution comprising sulphuric acid, DMF, iron(III) sulfate, and optionally an organic acid, in particular acetic acid.

The reaction of the compound of formula (III) with the haloacetonitrile is advantageously carried out under mild conditions, for example at a temperature of from 0 to 70° C., preferably of from 0° C. to room temperature and in particular of from 0 to 10° C. In general, the strong mineralic acid is added to the mixture of the reactants, solvent(s) and optional catalyst in a way that the temperature does not exceed the above-given limits. Following the addition of the mineral acid the reaction mixture, if necessary, may be kept at room temperature or at an elevated temperature, for example at a temperature from 50 to 100° C., preferably from 70 to 80° C., for a time suitable to complete the reaction, for example for up to 120 minutes and preferably for 30 to 90 minutes. Finally the reaction mixture is worked up in an usual manner, for example by hydrolyzing the reaction mixture with water at ambient conditions, washing, separating and drying the organic residue, thus obtaining the compound of formula (I), wherein R and R′ are each methyl.

The compounds of formula (I), wherein R and R′ are each hydrogen are known, for example, from A. Jirgensons et al., Synthesis 2000, 12, 1709 and may be obtained, for example, as described therein.

The reaction of the compounds of formula (I) with thiourea is advantageously carried out in an appropriate solvent at a temperature of, for example, from 60 to 120° C., preferably at reflux temperature of the reaction mixture, within a time period of, for example, from about 4 hours to about 10 hours. A suitable solvent is, for example a C₁-C₄-alcohol, for example methanol, ethanol or n- or isopropanol, in particular ethanol or isopropanol. The resulting compounds of formula (II) may be isolated in conventional manner, for example by removal of the solvent, washing, crystallizing and/or drying.

Thus, the present invention is directed to a process as described above, wherein reaction step (i) is carried out in a C₁-C₄-alcohol as a solvent.

A further object of the present invention is a compound of formula (II)

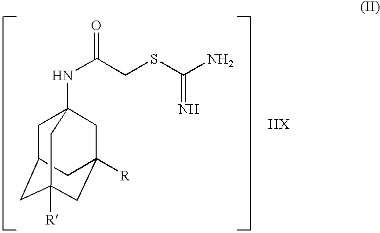

wherein R and R′ are each methyl, and X is chlorine or bromine.

Compounds of formula (II) are subsequently treated in an acidic medium in order to obtain the desired adamantanamine or a hydrohalogenid thereof. A preferred acidic medium is, for example, an aqueous acidic medium or a mixture of a C₁-C₄-alcohol and an acid or a mixture of a C₁-C₄-alcohol, water and an acid. A suitable acid in the conversion step of the compound of formula (II) is, for example, an organic C₁-C₄-carboxylic acid, for example acetic acid, propionic acid or butanoic acid, in particular acetic acid. Useful alcohols are as defined above, for example ethanol or isopropanol. In general, the compound of formula (II) is treated in the acidic medium under reflux for a time period sufficient to complete the conversion, which is, for example, a time period of up to 12 hours and preferably from 4 to 8 hours.

Thus, the present invention also refers to process as defined above, wherein the acid treatment in step (ii) comprises treating the compound of Formula (II) in a medium comprising a C₁-C₄-carboxylic acid, in particular acetic acid, and one or more solvents selected from the group consisting of water and a C₁-C₄-alcohol.

The step of isolating the compound of formula (II) may be omitted, and the compounds of formula (I) may be converted directly to the desired adamantanamine or a hydrohalogenid thereof by a treatment of the compound of formula (I) with thiourea, for example, under reflux in a reaction medium as described above, in particular in a reaction medium comprising an acid as described above, water and/or a C₁-C₄-alcohol.

The resulting adamantanamine in each case may be isolated from the reaction mixture in form of its free base, for example, by making the reaction solution alkaline and isolating and purifying the resulting product in known manner. For example, the reaction mixture is treated with an alkali hydroxide such as sodium hydroxide. Following the addition of a suitable organic solvent, a phase separation may be performed, and the organic layer then may be subjected to typical finishing steps such as concentration, crystallization, and/or drying steps.

Thus, the present invention refers to a process according to the above defined process, wherein in step (iii) the adamantanamine is isolated as the free base by making the reaction solution alkaline.

The adamantanamine in form of its free base may be easily converted into the respective hydrohalogenide by a treatment with the respective hydrohalogenic acid in a medium comprising, for example a C₁-C₄-alcohol and optionally water.

Therefore, the present invention is also directed to a process as described above which comprises as an additional step the conversion of the free adamantanamine base to an adamantanamine hydrohalogenid by a treatment with a hydrohalogenic acid, in particular with hydrochloric acid.

The preferred adamantanamines, Memantine hydrochloride or Amantadine hydrochloride, are advantageously obtained by treating Memantine or Amantadine in a solution comprising a C₁-C₄-alcohol, in particular ethanol or isopropanol, and aqueous hydrochloric acid. Preferably, concentrated hydrochloric acid is added slowly to the solution of Memantine or Amantadine in a C₁-C₄-alcohol.

Instead of isolating Memantine or Amantadine in form of its free base, the above reaction mixture comprising the raw Memantine or Amantadine may be converted directly to the respective Memantine or Amantadine hydrohalogenide without isolation of the free base by adding the hydrohalogenic acid directly to the reaction mixture and isolating the Memantine or Amantadine hydrohalogenide, for example, as described above.

Thus, the present invention also relates to a process as described above, wherein the compound of formula (I) is converted directly to the adamantanamine without isolation of the compound of formula (II) by reaction with thiourea in a medium comprising a C₁-C₄-carboxylic acid and one or more solvents selected from the group consisting of water and a C₁-C₄-alcohol. Further on, the present invention is also directed to such a process, wherein the adamantanamine is converted directly to the adamantanamine hydrohalogenide without isolation of the free base by adding the hydrohalogenic acid to the reaction mixture comprising the raw adamantanamine.

The examples further illustrate the present invention.

### EXAMPLE 1

### Preparation of 2-Chloro-N-(3,5-dimethyl-adamantan-1-yl)-acetamide (Method 1)

A 0.50 l round-bottom three-necked flask, equipped with reflux condenser, oil ventile, mechanical stirrer, thermometer, and dropping funnel is charged with 1-hydroxy-dimethyl-adamantane (10.00 g, 55.47 mmol), chloroacetonitrile (8.38 g, 110.94 mmol), and acetic acid (17.74 ml, 307.48 mmol). The resulting suspension is cooled to 0° C. with vigorous stirring. To the cold reaction mixture is added sulphuric acid (96%, 17.74 ml, 332.80 mmol), keeping the temperature below 10° C. After completion of addition the clear reaction mixture is allowed to reach room temperature. The resulting suspension is slowly treated with water (0° C., 100 ml) and stirred for further 30 min at 0-5° C. After separation, the colourless crystalline solid is washed in a stream of water (0° C., 100 ml, 2×) and dried under reduced pressure (40° C., 72 h, 25 mbar) to give 13.25 g (51.80 mmol, 93.4%) of 2-chloro-N-(3,5-dimethyl-adamantan-1-yl)-acetamide. ¹H NMR (300 MHz, DMSO-d6, ppm): δ 7.66 (b, 1H, NH), 3.93 (s, 2H, RCH₂Cl), 2.08 (m, 1H, R₃CH), 1.75 (m, 2H, RCH₂R), 1.57 (m, 4H, RCH₂R), 1.28 (m, 4H, RCH₂R), 1.11 (s, 2H, RCH₂R), 0.82 (s, 6H, RCH₃). ¹³C NMR (75 MHz, DMSO-d6, ppm): δ 164.82 (CO), 52.73, 50.12, 46.69, 43.42, 42.17, 31.81, 29.43. MS (El, m/z): 255 [M]⁺, 220 [M-Cl]⁺, 163 [M-C₂H₃ClNO]⁺. IR (KBr, cm⁻¹): 1661 s (ν_{RCONHR}, Amide I), 1574 s (Amide II). E.A. (C H N, %): calc. for C₁₄H₂₂ClNO 0.1 HAc: C, 65.15; H, 8.00; N, 5.15. Found: C, 65.15; H, 8.62; N, 5.35. mp.: 106.8° C.

### EXAMPLE 2

### Preparation of 2-Chloro-N-(3,5-dimethyl-adamantan-1-yl)-acetamide (Method 2)

A 4.0 l round-bottom three-necked flask, equipped with reflux condenser, oil ventile, mechanical stirrer, thermometer, and dropping funnel is charged with 1-hydroxy-dimethyl-adamantane (47.50 g, 263.48 mmol), dimethylformamide (42.13 ml), chloroacetonitrile (39.81 g, 526.97 mmol), and acetic acid (84.27 ml, 1.46 mol). The resulting suspension is slowly treated with sulphuric acid (96%, 84.27 ml, 1.58 mol) at room temperature with vigorous stirring upon which the reaction mixture reaches approximately 70° C. After completion of addition the clear reaction mixture is allowed to cool to room temperature. The resulting suspension is slowly treated with water (0° C., 475 ml) and stirred for further 60 min at 0-5° C. After separation, the colourless crystalline solid is washed in a stream of water (0° C., 475 ml, 2×) and dried under reduced pressure (40° C., 72 h, 25 mbar) to give 62.18 g (243.09 mmol, 92.26%) of 2-chloro-N-(3,5-dimethyl-adamantan-1-yl)-acetamide.

### EXAMPLE 3

### Preparation of 2-Chloro-N-(3,5-dimethyl-adamantan-1-yl)-acetamide (Method 3)

A 0.50 l round-bottom three-necked flask, equipped with reflux condenser, oil ventile, mechanical stirrer, thermometer, and dropping funnel is charged with 1-hydroxy-dimethyl-adamantane (10.00 g, 55.47 mmol), chloroacetonitrile (8.38 g, 110.94 mmol), dimethyl-formamide (8.87 ml) and acetic acid (17.74 ml, 307.48 mmol). The resulting suspension is slowly treated with sulphuric acid (96%, 17.74 ml, 332.80 mmol) at room temperature with vigorous stirring upon which the reaction mixture reaches approximately 70° C. After completion of addition the clear reaction mixture is slowly treated with water (15° C., 100 ml) at 70° C. Upon ceasing of the exothermic reaction the product crystallizes. The resulting suspension is cooled to 0-5° C. and stirred for further 60 min at this temperature. After filtration, the colourless crystalline solid is washed in a stream of water (20° C., 50 ml, 4×) and dried under reduced pressure (40° C., 72 h, 25 mbar) to yield 13.71 g (53.59 mmol, 96.61%) of 2-chloro-N-(3,5-dimethyl-adamantan-1-yl)-acetamide.

### EXAMPLE 4

### Preparation of 2-Chloro-N-(3,5-dimethyl-adamantan-1-yl)-acetamide (Method 4)

A 0.50 l round-bottom three-necked flask, equipped with reflux condenser, oil ventile, mechanical stirrer, thermometer, and dropping funnel is charged with 1-hydroxy-dimethyl-adamantane (10.00 g, 55.47 mmol), chloroacetonitrile (8.38 g, 110.94 mmol), and acetic acid (17.74 ml, 307.48 mmol). The resulting suspension is slowly treated with sulphuric acid (96%, 17.74 ml, 332.80 mmol) at room temperature with vigorous stirring upon which the reaction mixture reaches approximately 70° C. After completion of addition the clear reaction mixture is slowly treated with water (15° C., 100 ml) at 70° C. Upon ceasing of the exothermic reaction the product crystallizes. The resulting suspension is cooled to 0-5° C. and stirred for further 60 min at this temperature. After filtration, the colourless crystalline solid is washed in a stream of water (20° C., 50 ml, 4×) and dried under reduced pressure (40° C., 72 h, 25 mbar) to yield 13.61 g (53.19 mmol, 95.89%) of 2-chloro-N-(3,5-dimethyl-adamantan-1-yl)-acetamide.

### EXAMPLE 5

### Preparation of 2-Chloro-N-(3,5-dimethyl-adamantan-1-yl)acetamide (Method 5)

A 0.25 l round-bottom three-necked flask, equipped with reflux condenser, oil ventile, mechanical stirrer, thermometer, and dropping funnel is charged with 1-bromo-3,5-dimethyladamantane compound (5.00 g, 20.56 mmol), iron(III)sulphate (84 mg, 0.21 mmol), dimethylformamide (6.00 ml), chloroacetonitrile (4.19 g, 55.50 mmol), and acetic acid (9.23 g, 153.73 mmol). The reaction mixture is cooled to 5-10° C. with stirring. Sulphuric acid (96%, 8.87 ml, 166.4 mmol) is added drop wise, keeping the temperature at 5-10° C. After completion of addition the resulting suspension is heated to 80° C. for about 90 min. The reaction mixture is allowed to cool to room temperature and hydrolyzed with water (50 ml). After cooling to 5° C. and stirring for further 30 min. the residue is separated and washed in a stream of water (150 ml) and dried under reduced pressure (40° C., 72 h, 25 mbar) to yield 3.23 g (12.63 mmol, 61.4%) of 2-chloro-N-(3,5-dimethyl-adamantan-1-yl)-acetamide as a colourless powder as described above.

### EXAMPLE 6

### Preparation of 2-Chloro-N-(3,5-dimethyl-adamantan-1-yl)acetamide (Method 6)

A 0.25 l round-bottom three-necked flask, equipped with reflux condenser, oil ventile, mechanical stirrer, thermometer, and dropping funnel is charged with 1-bromo-3,5-dimethyladamantane (5.00 g, 20.56 mmol), iron(III)sulphate (84 mg, 0.21 mmol) and dimethylformamide (6.00 ml). The reaction mixture is cooled to 5-10° C. with stirring. Sulphuric acid (96%, 8.87 ml, 166.4 mmol) is added drop wise, keeping the temperature at 5-10° C. After completion of addition the resulting suspension is treated with chloroacetonitrile (4.19 g, 55.50 mmol) at 5° C. and then heated to 70-80° C. for 30 min. The reaction mixture is allowed to cool to room temperature and hydrolyzed with water (50 ml). After cooling to 5° C. and stirring for further 30 min. the residue is separated and washed in a stream of water (150 ml) and dried under reduced pressure (40° C., 72 h, 25 mbar) to yield 3.52 g (13.76 mmol, 66.9%) of 2-chloro-N-(3,5-dimethyl-adamantan-1-yl)-acetamide as described above.

### EXAMPLE 7

### Preparation of 2-Carbamimidosulfanyl-N-(3,5-dimethyl-adamantan-1-yl)acetamide

A 0.25 l round-bottom two-necked flask, equipped with reflux condenser, oil ventile and magnetic stir bar is charged with 2-chloro-N-(3,5-dimethyl-adamantan-1-yl)-acetamide (2.00 g, 7.82 mmol), ethanol (16 ml) and thiourea (718 mg, 9.43 mmol). The suspension is heated to reflux with stirring. After 10 min. the solvent is removed under reduced pressure resulting in a colourless oil. Upon addition of acetonitrile (76 ml) with stirring the residue crystallizes. The resulting suspension is cooled to 0° C. and stirring is continued for further 60 min. The colourless precipitate is separated, washed with acetonitrile (10 ml, 3×) and dried under reduced pressure (40° C., 60 mbar, 8 h) to yield 1.96 g (5.90 mmol, 75.5%) of 2-carbamimidosulfanyl-N-(3,5-dimethyl-adamantan-1-yl)acetamid as a colourless powder. ¹H NMR (300 MHz, DMSO-d6, ppm): 9.52 (b, 1H, NH), 9.27 (b, 1H, NH), 3.90 (s, 2H, RCH₂S), 2.08 (m, 1H, R₃CH), 1.73 (m, 2H, RCH₂R), 1.57 (m, 4H, RCH₂R), 1.28 (m, 4H, RCH₂R), 1.09 (s, 2H, RCH₂R), 0.80 (s, 6H, RCH₃). ¹³C NMR (75 MHz, DMSO-d6, ppm): δ 170.13, 166.69 (CO), 53.21, 50.05, 46.49, 42.09, 34.07, 31.77, 29.92, 29.35. MS (ESI, m/z): 296.0 [M-Cl]⁺. IR (KBr, cm⁻¹): 1663 s (V_{RCONHR}, Amide I), 1553 s (Amide II). mp.: 212.3° C. (dec.). E.A. (C, H, N; %): calc. for C₁₅H₂₆ClN₃OS: C, 54.18; H, 7.90; N, 12.66. Found: C, 54.11, H, 6.94, N, 12.56.

### EXAMPLE 8

### Preparation of 1-Amino-3,5-dimethyladamantan (Memantine base)

A 0.50 l round-bottom three-necked flask, equipped with reflux condenser, oil ventile, mechanical stirrer, thermometer, and dropping funnel is charged with 2-chloro-N-(3,5-dimethyl-adamantan-1-yl)-acetamide (10.00 g, 39.10 mmol), ethanol (77.52 ml), acetic acid (15.5 ml), and thiourea (3.57 g, 46.92 mmol). The resulting suspension is heated to reflux (70° C.) and held at this temperature for 6 h with stirring. The turbid solution is made alkaline with sodium hydroxide (50%, 15.5 ml) and treated with toluene at approximately 60° C. A phase separation is performed, the aqueous layer is discarded. The organic layer is concentrated under vacuum, dissolved in iso-propanol (5 ml) and filtered. The residue is dried to give 5.22 g (29.12 mmol, 74.5%) of 1-amino-3,5-dimethyladamantan as a liquid, which crystallizes upon storage. ¹H NMR (300 MHz, DMSO-d6, ppm): δ 2.04 (m, 1H, R₃CH, 1.45 (b, 1.5H, RNH₂), 1.33 (m, 2H, RCH₂R), 1.25-0.98 (m, 10H, RCH₂R), 0.80 (s, 6H, RCH₃). ¹³C NMR (75 MHz, DMSO-d6, ppm): δ 52.31, 50.39, 48.39, 44.43, 42.37, 32.16, 30.13, 30.00. MS (El, m/z): 179 [M]⁺. IR (film, cm⁻¹): 3342, 3268 cm⁻¹ (m, ν_{NH}), 1593 (m, δ_{NH}).

### EXAMPLE 9

### Preparation of 1-Amino-3,5-dimethyladamantan Hydrochloride (Memantine hydrochloride) (Method 1)

In a 0.05 l round-bottom flask equipped with magnetic stir bar, nitrogen inlet and oil ventile 1-amino-3,5-dimethyladamantan (2.69 g, 15.00 mmol) is dissolved in iso-propanol (2.7 ml). To the stirred clear solution aq. hydrochloric acid (31%, 2 ml) is added drop wise at room temperature. The resulting suspension is cooled to 0° C. and stirred for further 60 min. The colourless crystalline solid is collected by filtration, washed with iso-propanol (0.5 ml, 3×) and dried under reduced pressure (48 h, 30° C., 50 mbar) to give 2.24 g (10.38 mmol, 69.2%) of Memantine hydrochloride. ¹H NMR (300 MHz, DMSO-d6, ppm): δ 8.24 (s, 3H, NH), 2.11 (m, 1H, R₃CH), 1.65 (m, 2H, RCH₂R), 1.46 (m, 4H, RCH₂R), 1.28 (s, 4H, RCH₂R), 1.10 (m, 2H, RCH₂R) 0.83 (s, 6H, RCH₃). ¹³C NMR (75 MHz, DMSO-d6, ppm): δ 52.81, 49.96, 46.31, 41.96, 38.91, 32.37, 30.05, 29.54. MS (El, m/z, free base): 179 [M]⁺. IR (KBr, cm⁻¹): 3435 (m, ν_{NH}). mp.: >300 (subl.). E.A. (C, H, N; %): calc. for C₁₂H₂₂ClN.H₂O: C, 66.25; H, 10.28; N, 6.44. Found: C, 66.31%; H, 9.40; N, 6.20. GC (rel. Ar.): 99.85%.

### EXAMPLE 10

### Preparation of 1-Amino-3,5-dimethyladamantan Hydrochloride (Memantine hydrochloride) (Method 2)

A 0.50 l round-bottom flask equipped with reflux condenser, oil ventile, mechanical stirrer, thermometer and dropping funnel is charged with 2-chloro-N-(3,5-dimethyl-adamantan-1-yl)-acetamide (10.00 g, 39.10 mmol), thiourea (3.571 g, 46.92 mmol), water (77.5 ml), and acetic acid (15.5 ml) is heated to reflux and held at this temperature for 6 h with stirring. The reaction mixture is allowed to cool to approximately 60° C. and is treated drop wise with aq. hydrochloric acid (31%, 25 ml). The resulting suspension is cooled to room temperature. The precipitate is collected by filtration, washed with water (10 ml) and dried under reduced pressure (30° C., 25 mbar, 12 h) to give 5.78 g (26.87 mmol, 68.7%) of Memantine hydrochloride as a colourless crystalline solid as described in example 9.

### EXAMPLE 11

### Preparation of 1-Amino-3,5-dimethyladamantan Hydrochloride (Memantine hydrochloride) (Method 3)

A 4.0 l round-bottom three-necked flask, equipped with reflux condenser, oil ventile, mechanical stirrer, thermometer, and dropping funnel is charged with 2-chloro-N-(3,5-dimethyl-adamantan-1-yl)-acetamide (106.7 g, 0.42 mol), thiourea (38.1 g, 0.50 mol), acetic acid (165.4 ml), and ethanol (827.1 ml). The reaction mixture is heated to reflux with stirring and held for 8 h at this temperature. After completion of the reaction the vessel contents are allowed to reach room temperature. The turbid solution is filtered and the clear filtrate is treated first with water (1070 ml), then with aq. sodium hydroxide (50%, 165.4 ml) without external cooling in a period of 15 min. Temperature may reach up to 60° C. Toluene (215 ml) is added and the mixture is vigorously stirred for 15 min. A phase separation is performed. The aqueous phase is extracted once with toluene (220 ml) at 45-55° C. The phases are separated, organic layers are combined, aqueous layers discarded. To the combined organic phases sodium hydroxide is added (50%, 100 ml) and the two phase system is vigorously stirred at 45-55° C. for 15 min. The phases are separated, the aqueous layer is discarded. The organic layer is treated with water (100 ml), making sure the temperature is maintained between 45-55° C. The phases are separated, the aqueous layer is discarded. The organic layer is treated with sodium chloride (aq., conc, 100 ml) with stirring, maintaining the temperature between 45-55° C. A phase separation is performed, the aqueous layer is disposed. The organic phase is filtered and transferred to a 2.0 l round-bottom three-necked flask, equipped with reflux condenser, oil ventile, mechanical stirrer, thermometer, and dropping funnel. The vessel contents are cooled to 0-5° C. with stirring. Aq. hydrochloric acid (31%, 53 ml) is added, keeping the temperature below 20° C. After completion of addition, the resulting suspension is cooled to 0-5° C. and stirred for further 30 min. The colourless, microcrystalline solid is collected by filtration, washed with toluene (100 ml, 3×) and dried under reduced pressure (40° C., 30 mbar, 18.5 h) to give 71.25 g (0.33 mol, 71.16%) of Memantine hydrochloride as crystalline solid as described in example 9.

### EXAMPLE 12

### Synthesis of N-Adamantan-1-yl-2-chloro-acetamide

A 0.50 l round-bottom three-necked flask, equipped with reflux condenser, oil ventile, mechanical stirrer, thermometer, and dropping funnel is charged with 1-hydroxyadamantane (10.00 g, 65.69 mmol), chloroacetonitrile (9.92 g, 131.38 mmol), dimethylformamide (11 ml) and acetic acid (21 ml, 363.96 mmol). The resulting suspension is slowly treated with sulphuric acid (96%, 21 ml) at room temperature with vigorous stirring upon which the reaction mixture reaches approximately 80° C. After completion of addition the clear reaction mixture is slowly treated with water (15° C., 118 ml) at 70° C. Upon ceasing of the exothermic reaction the product crystallizes. The resulting suspension is cooled to 0-5° C. with stirring. After filtration, the colourless crystalline solid is washed in a stream of water and dried under reduced pressure (40° C., 19 h, 25 mbar) to yield 14.90 g (65.43 mmol, 99.60%) of N-Adamantan-1-yl-2-chloro-acetamide. ¹H NMR (300 MHz, CDCl₃, ppm): 6.24 (b, 1H, NH), 3.94 (s, 2H, RCH₂Cl), 2.10 (m, 3H, R₃CH), 2.03 (m, 6H, RCH₂R), 1.70 (m, 6H, RCH₂R). ¹³C NMR (75 MHz, DMSO-d6, ppm): δ 164.61 (CO), 52.39, 42.89, 41.37, 36.22, 29.38. MS (El, m/z): 227 [M]⁺. IR (KBr, cm⁻¹): 1662 s (ν_{RCONHR}, Amide I), 1569 s (Amide II). mp.: 123.4° C.

### EXAMPLE 13

### Prep. of N-Adamantan-1-yl-2-carbamimidoylsulfanyl-acetamide Hydrochloride

A 0.25 l round-bottom two-necked flask, equipped with reflux condenser, oil ventile and magnetic stir bar is charged with N-adamantan-1-yl-2-chloro-acetamide (2.00 g, 8.78 mmol), ethanol (16 ml) and thiourea (802 mg, 10.54 mmol). The suspension is heated to reflux with stirring. After 15 min. the solvent is removed under reduced pressure. Acetonitrile (76 ml) is added to the oily residue with stirring. The resulting suspension is stirred for further 30 min. The colourless precipitate is separated at room temperature, washed with acetonitrile (10 ml, 3×) and dried under reduced pressure (40° C., 60 mbar, 19 h) to yield 2.18 g (7.16 mmol, 81.6%) of the title compound as colourless, microcrystalline powder. ¹H NMR (300 MHz, DMSO-d6, ppm): 9.42 (b, 3H, NH), 8.32 (s, 1H, NH), 3.95 (s, 2H, RCH₂S), 2.07-1.81 (m, 9H, R₃CH, RCH₂R), 1.61 (s, 6H, RCH₂R). ¹³C NMR (75 MHz, DMSO-d6, ppm): 6170.18, 166.65 (CO), 51.67, 40.57, 35.84, 34.14, 28.70. MS (ESI, m/z): 298.0 [MH—Cl]⁺. IR (KBr, cm⁻¹): 1662 s (ν_{RCONHR}, Amide I), 1555 s (Amide II). mp.: 180° C. (dec.). E.A. (C, H, N; %): calc. for C₁₅H₂₆ClN₃OS 0.1H₂O: C, 51.08; H, 7.32; N, 13.75. Found: C, 51.07; H, 7.22; N, 13.65.

### EXAMPLE 14

### Synthesis of Amantadine HCl

A 2.0 l round-bottom three-necked flask, equipped with reflux condenser, oil ventile, mechanical stirrer, thermometer, and dropping funnel is charged with N-adamantan-1-yl-2-chloro-acetamide (71.0 g, 0.31 mol), thiourea (28.5 g, 0.37 mol), acetic acid (123.6 ml), and ethanol (550.2 ml). The reaction mixture is heated to reflux with stirring and held for 8 h at this temperature. After completion of the reaction the vessel contents are allowed to reach room temperature. The suspension is filtered over cellites and the clear filtrate is treated first with water (710 ml), then with aq. sodium hydroxide (50%, 124 ml) without external cooling in a period of 10 min. Temperature may reach up to 50° C. Toluene (142 ml) is added and the mixture is vigorously stirred for 15 min. A phase separation is performed. The aqueous phase is extracted once with toluene (142 ml) at 45-55° C. The phases are separated, organic layers are combined, aqueous layers discarded. To the combined organic phases sodium hydroxide is added (50%, 71 ml) and the two phase system is vigorously stirred at 45-55° C. for 15 min. The phases are separated, the aqueous layer is discarded. The organic layer is diluted with toluene (20 ml) and washed with sodium hydroxide (50%, 71 ml) at 50-60° C., aqueous layers are discarded. The organic layer is treated with water (71 ml), making sure the temperature is maintained between 50-60° C. The phases are separated, the aqueous layer is discarded. The organic layer is treated with sodium chloride (aq., sat., 71 ml) with stirring, maintaining the temperature between 45-55° C. A phase separation is performed, the aqueous layer is disposed. The organic phase is filtered over cellite and transferred to a 2.0 l round-bottom three-necked flask, equipped with reflux condenser, oil ventile, mechanical stirrer, thermometer, and dropping funnel. Transfer lines are flushed with toluene (20 ml, 2×). The vessel contents are cooled to 0-5° C. with stirring. Aq. hydrochloric acid (31%, 53 ml) is added, keeping the temperature below 20° C. After completion of addition, the resulting suspension is cooled to 0-5° C. and stirred for further 60 min. The colourless, microcrystalline solid is collected by filtration, washed with toluene and dried under reduced pressure (40° C., 30 mbar, 18.5 h) to give 47.34 g (0.25 mol, 80.89%) of Amantadine HCl as microcrystalline solid. mp. >300° C. (subl.). ¹H NMR (300 MHz, DMSO-d6, ppm): δ 7.24 (b, 3H, NH), 2.06 (m, 3H, R₃CH), 1.82 (s, 6H, RCH₂R), 1.60 (m, 6H, RCH₂R).

1. A process for the manufacture of an adamantanamine of formula 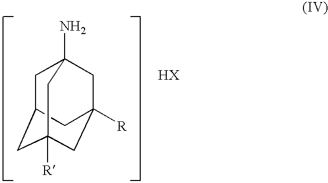
wherein R and R′ are each methyl and X is halogen, which comprises
(i) reacting a compound of formula 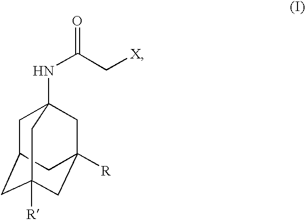
wherein R, R′ and X are each as defined above, with thiourea;
(ii) subjecting the resulting compound of formula 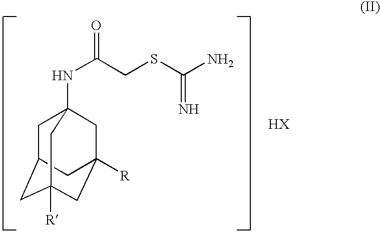
to an acid treatment and
(iii) isolating the resulting adamantanamine or a hydrohalogenide thereof.

2. A process according to claim 1, wherein reaction step (i) is carried out in a C₁-C₄-alcohol as a solvent.

3. A process according to claim 1, wherein the acid treatment in step (ii) comprises treating the compound of Formula (II) in a medium comprising a C₁-C₄-carboxylic acid, in particular acetic acid, and one or more solvents selected from the group consisting of water and a C₁-C₄-alcohol.

4. A process according to claim 1, wherein in step (iii) the adamantanamine is isolated as the free base by making the reaction solution alkaline.

5. A process according to claim 4, comprising as an additional step the conversion of the free adamantanamine base to an adamantanamine hydrohalogenid by a treatment with a hydrohalogenic acid, in particular with hydrochloric acid.

6. A process according to claim 1, wherein the compound of formula (I) is converted directly to the adamantanamine without isolation of the compound of formula (II) by reaction with thiourea in a medium comprising a C₁-C₄-carboxylic acid and one or more solvents selected from the group consisting of water and a C₁-C₄-alcohol.

7. A process according to claim 6, wherein the adamantanamine is converted directly to the adamantanamine hydrohalogenide without isolation of the free base by adding the hydrohalogenic acid to the reaction mixture comprising the raw adamantanamine.

8. A compound of formula 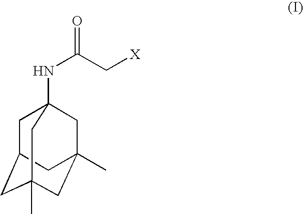
wherein X is chlorine or bromine.

9. A process for preparing a compound of formula (I) according to claim 8, wherein a compound of formula 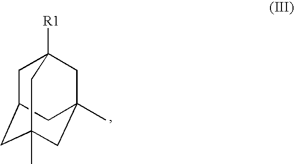
wherein R1 is hydroxy or halogen, is reacted with a haloacetonitrile X—CH₂—CN, wherein X is a halogen, in an acidic medium.

10. A compound of formula 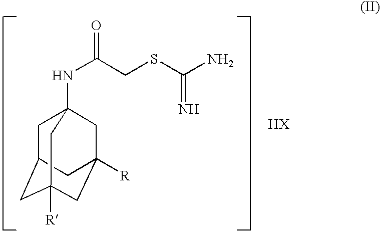
wherein R and R′ are each methyl, and X is chlorine or bromine.

11. Use of a compound of formula (I) according to claim 8 for the manufacture of Memantine or a hydrohalogenide thereof.

12. Use of a compound of formula (II) according to claim 10 for the manufacture of Memantine or a hydrohalogenide thereof.
